(19) United States
(12) Patent Application Publication
(10) Pub. No.: US 2003/0073730 A1
(43) Pub. Date: Apr. 17, 2003

(54) Use of amino-isoxazolidone compounds for treatment of memory impairment following traumatic brain injury

(75) Inventors: Daniel T. Fakouhi, Wilmette, IL (US); Robert L. Herting, Park Ridge, IL (US); J. Michael Williams, Philadelphia, PA (US)

Correspondence Address: J. Timothy Keane, PHARMACIA CORPORATION, Corporate Patent Department, 800 North Lindbergh Blvd. - O4E, St. Louis, MO, 63167, US

(73) Assignee: G.D. Searle & Co., Chicago, IL (US)

(21) Appl. No.: 10/271,814

(22) Filed: Oct. 16, 2002

Related U.S. Application Data
(63) Continuation of application No. 09/506,070, filed on Feb. 17, 2000.
Application No. 09/506,070 is a continuation of application No. 09/149,411, filed on Sep. 8, 1998, now abandoned.
Application No. 09/149,411 is a continuation of application No. 08/848,348, filed on May 20, 1997, now abandoned.
Application No. 08/848,348 is a continuation of application No. 08/344,033, filed on Nov. 23, 1994, now abandoned.

(51) Int. Cl. A61K 31/42

(52) U.S. Cl. 514/380

(57) ABSTRACT

A class of amino-isoxazolidone compounds is described for use in treatment of memory impairment associated with Traumatic Brain Injury (“TBI”). Preferred compounds of this class are D-cycloserine and its prodrugs.

### FIELD OF THE INVENTION

This invention is in the field of clinical neurology and relates specifically to compounds, formulations and methods for treatment of memory impairment or deficit following or associated with Traumatic Brain Injury (“TBI”).

### BACKGROUND OF THE INVENTION

In a summary of epidemiology of Traumatic Brain Injury (“TBI”) in the United States, an overall incidence of 200 per 100,000 people was reported [J. F. Kraus, In: Neuroepidemiology, 335-357, D. W. Anderson & D. H. Schoenberg, eds., CRC Press, Boca Raton, Fla., (1991)]. TBI patients may develop a number of neuropsychological disorders, including severe memory impairment. Epidemiological studies of cognitive impairment following Traumatic Brain Injury have not been reported. However, outcome studies using consecutive admissions to trauma centers suggest that the incidence rate leading to cognitive impairment is approximately 40-80% of all cases of Traumatic Brain Injury in which the patient was in a coma for at least one-half hour [J. M Williams et al, J. Neurosurg., 61, 581-585 (1984); M. R. Bond et al, Scand. J. of Rehab. Med., 8, 127-133 (1976); G. S. Carlsson et al, J. of Neurosurgery, 67, 507-513 (1986); S. Dikmen et al, Arch. Physical Medicine and Rehabilitation, 67, 507-513 (1986); S. Dikmen et al, J. Neurology, Neurosurgery and Psychiatry, 50, 1613-1618 (1987); H. S. Levin et al, J. Neurosurgery, 50, 412-422, (1979)] Post-traumatic memory impairment persists through the patient's physical recovery and may become the permanent sequelae of the injury [G. S. Carlsson et al, J. of Neurosurgery, 67, 507-513 (1986); S. Dikmen et al, Arch. Physical Medicine and Rehabilitation, 67, 507-513 (1986); S. Dikmen et al, J. Neurology, Neurosurgery and Psychiatry, 50, 1613-1618 (1987); H. S. Levin et al, J. Neurosurgery, 50, 412-422, (1979)).

Traumatic injury of the brain produces mechanical injury of neurons, axonal stretching and neuronal degeneration. Compression injury of neural tissues may also result from increased intracranial pressure due to hematoma and diffuse swelling.

Finally, significant anoxia and ischemia may occur because of mechanical and compression damage to the cerebral vasculature. The general result of these factors is decreased brain function, neurological symptoms and posttraumatic cognitive disorders [F. S. Vogel, In: G. L., Odom, ed. CNS Trauma Research Status Report, 114-122, NINCDS, Bethesda, Md. (1979)].

Post-traumatic cognitive disorders including memory impairment are caused in part by the disruption of a wide variety of neurotransmitter systems (F. S. Vogel, In: G. L. Odom, ed. CNS Trauma Research Status Report, 114-122, NINCDS, Bethesda, Md. (1979); A. I. Faden et al, Science, 244, 798-200 (1989)], causing significant disruption of glycine and glutamate metabolism that results in secretion of these transmitters at a toxic level. Mechanisms of injury include impaired function of the NMDA receptor site, down-regulation of NMDA receptors on surviving neurons, and disruption of presynaptic excitatory amino acid synthesis and release [B. S. Meldrum et al, In: J. C. Watkins and G. L. Collingridge, eds., The NMDA Receptor, Oxford University Press, New York (1989)]. It is hypothesized that the function of the NMDA receptor and glycine metabolism are affected by traumatic injury, and disruption of this neurotransmitter system contributes to the cognitive impairment commonly associated with these injuries.

Cycloserine has been shown to freely cross the blood-brain barrier [K. G. S. Nair et al, In: Antibiotics Annual, 169-172, Med Encyclopedia Inc., New York (1955)] and act as a potent and selective modulator of the NMDA receptor-associated glycine recognition site [G. B. Watson et al, Brain Resarch, 510, 158-160 (1990)]. Activation of the NMDA receptors by cycloserine is suggested as a possible mechanism for its positive effect on memory consolidation and retrieval process in animal models G. E. Handelman et al, Society for Neuroscience Abstracts, 14, (1), 249 (1988)]; J. B. Monahan et al, Pharmacology, Biochemistry and Behavior, 34, 649-653, (1989)]. Cycloserine is a partial agonist for the glycine-B site of the NMDA receptor. This means it will be stimulatory when glycine concentrations are low but will serve as an antagonist in the presence of glycine excess. Thus, hyperstimulation of the NMDA receptor with possible neurotoxicity will be prevented by cycloserine. Such action will confer additional safety to patients who may be experiencing hyperstimulation for other reasons at the time of treatment.

Recent studies in normal young and elderly human volunteers have shown that cycloserine in single oral doses of 5 and 15 mg can ameliorate some memory deficits induced by scopolamine [G. D. Searle & Co., Skokie, Ill., Protocol No.: EC6-88-02-001; Report No.: EC6-90-06-001, Sep. 14, 1990, as amended Jul. 10, 1991]. In addition, in the absence of scopolamine, cycloserine in the same dose range had significant positive effects on the cognitive parameters in the elderly population [G. D. Searle & Co., Skokie, Ill.; Protocol No.: EC6-89-02-002; Report No.: EC6-91-06-002, Jul. 10, 1991, as amended Aug. 12, 1991].

Amino-oxazolidone compounds have been investigated for CNS effects. For example, the compound D-cycloserine, in its D- and L-isomer forms, has been evaluated for CNS effects in animals [0. Mayer et al, Arzneim. Forsch., 21(2), 298-303 (1971)]. These cycloserine isomers have also been evaluated for psychological and physiological effects in human subjects. For example, D-cycloserine when administered at 500 mg/day doses to healthy human subjects, appeared to stimulate slight sociability, but with depressed mental alertness [M. Vojtechovsky, Act. Nerv. Super., 7(3), 269 (1965)]. Also, D-cyloserine has been administered at 1000 to 1500 mg/day to healthy volunteers whose blood levels showed increased levels of monoamine oxidase enzyme activity [V. Vitek et al, Psychopharmacologia, 7(3), 203-219 (1965)]

D-cycloserine has been investigated as a therapeutic agent for mental disorders in clinical trials, wherein D-cycloserine was administered to mentally disturbed patients at doses of 500 mg per day [G. E. Crane, Compr. Psychiat., 2, 51-53 (1961)]. In such clinical trials, improvements in depression, insomnia, anexoria or tension were found for some patients, while patients suffering from severe neurosis or psychosis responded poorly to such medication. Moreover, D-cycloserine has been used to exacerbate the symptoms of schizophrenia in an attempt to cure the ailment by symptom provocation [J. Simeon et al, Compr. Psychiat., 11, 80-88, (1970)]. It appears that D-cycloserine, at the dose levels used in these studies, is acting as an antagonist at the glycine site of the NMDA-PCP receptor complex mimicking the action of PCP by inducing psychosis.

Other CNS-related investigations have been conducted with amino-oxazolidone compounds for interactions with the NMDA receptor complex. For example, U.S. Pat. No. 4,904,681 issued on Feb. 27, 1990 to A. A. Cordi et al describes evaluation of D-cycloserine as a glycine B partial agonist interacting with the NMDA receptor complex for treatment of learning or memory dysfunctions or for enhancement of cognitive functions. U.S. Pat. No. 5,061,721 isued on Oct. 29, 1991 to A. A. Cordi et al describes methods of treating Alzheimer's Disease, age-associated memory impairment, learning deficit and psychotic disorders, as well as methods for improving memory or learning in healthy subjects, using a composition containing a mixture of D-cycloserine and D-alanine. U.S. Pat. No. 5,087,633 issued on Feb. 11, 1992 to A. A. Cordi et al describes use of a glycine B partial agonist for memory and learning enhancment or treatment of a cognitive disorder. U.S. Pat. No. 5,187,171 issued on Feb. 16, 1993 to A. A. Cordi describes D-cycloserine as a glycine B partial agonist interacting with the NMDA receptor complex for treatment of psychotic conditions. U.S. Pat. No. 5,260,324 issued on Nov. 9, 1993 to A. A. Cordi et al describes compositions containing D-cycloserine with a side-effecting reducing amount of D-alanine, in a ratio range of about 1-to-1 to about 100-to-1 of D-alanine to D-cycloserine, for use in learning-memory enhancement or treatment of a cognitive psychotic disorder. U.S. patent application Ser. No. 08/155,986 filed on Nov. 22, 1993 of M. Nevins describes use of D-cylcoserine for treatment of anxiety.

### BRIEF DESCRIPTION OF THE DRAWINGS

FIG. 1 shows evaluation on a Memory Assessment Scale of visual memory score regression of endpoint score on baseline by treatment with D-cycloserine.

### DESCRIPTION OF THE INVENTION

Treatment of a memory impairment or deficit following or associated with Traumatic Brain Injury (“TBI”), is achieved by administering to a subject, susceptible to or suffering from such TBI-associated memory impairment or deficit, a therapeutically-effective amount of a glycine B partial agonist. A glycine B partial agonist for such treatment may be provided by one or more amino-isoxazolidone compounds, or a prodrug thereof, selected from a family of compounds of Formula I:

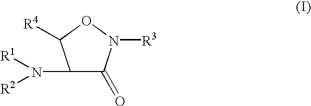

wherein R¹ is selected from hydrido, alkyl, haloalkyl, alkoxyalkyl, cycloalkyl, aralkyl and aryl; wherein each of R² and R³ is independently selected from hydrido, alkyl, aralkyl, aryl,

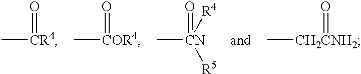

wherein R¹ and R² may be taken together to form a Schiff-base derived group selected from derivatives of aldehydes and ketones; wherein each of R⁴ and R⁵ is independently selected from hydrido, alkyl, haloalkyl, alkoxyalkyl, cycloalkyl, aralkyl and aryl; or a pharmaceutically-acceptable salt thereof. Where compounds of Formula I exist as optical isomers, the D-configuration is generally preferred.

A preferred family of compounds consists of compounds wherein R¹ is selected from hydrido, lower alkyl, haloalkyl, cycloalkyl, alkoxyalkyl, phenalkyl and phenyl; wherein each of R² and R³ is independently selected from hydrido, lower alkyl, phenalkyl, phenyl,

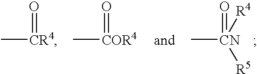

wherein the Schiff-base derived group is derived from acetylacetone, salicylaldehyde, benzophenone derivatives and acetylacetic acid esters; and wherein each of R⁴ and R⁵ is independently selected from hydrido, lower alkyl, phenyl and benzyl.

A more preferred group of compounds within Formula I consists of these compounds wherein R¹ is hydrido; wherein each of R² and R³ is independently selected from

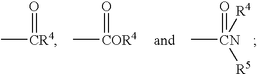

wherein the Schiff-base derived group is selected from

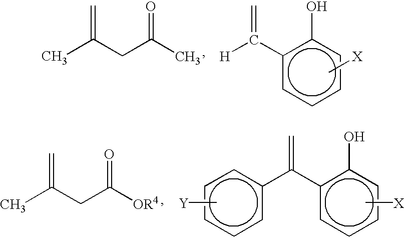

wherein each of X and Y is independently selected from one or more groups, substitutable at a substitutable position, selected from hydrido, lower alkyl and halo; and wherein each of R⁴ and R⁵ is independently selected from hydrido, lower alkyl and phenyl.

A most preferred group of compounds within Formula I consists of those compounds wherein R¹ is hydrido; wherein the Schiff-base derived group is selected from

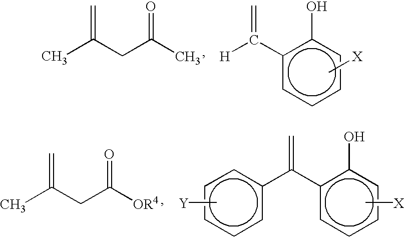

wherein each of X and Y is independently selected from fluoro, chloro and bromo; and wherein each of R², R³ and R⁴ is hydrido.

A most preferred specific compound of Formula I is the compound 4-amino-3-isoxazolidone having the structural formula

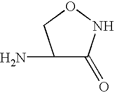

This compound exists in the L- and D-isomeric forms, of which the compound D-cycloserine is most highly preferred.

Also embraced by Formula I are the tautomeric forms of these compounds as represented by Formula II:

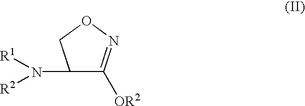

wherein R¹, R² and R³ are as defined for the compounds of Formula I.

The term “hydrido” denotes a single hydrogen atom (H) which may be attached, for example, to a carbon atom to form a hydrocarboyl group (—CH—); or the hydrogen atom may be attached attached to an oxygen atom to form a hydroxyl group (—OH). Where the term “alkyl” is used, either alone or within another term such as “haloalkyl”, the term “alkyl” embraces linear or branched radicals having one to about twenty carbon atoms or, preferably, one to about ten carbon atoms. More preferred alkyl radicals are “lower alkyl” radicals having one to about five carbon atoms. The term “cycloalkyl” embraces cyclic radicals having three to about ten ring carbon atoms, and preferably having three to about five carbon atoms, such as cyclopropyl and cyclobutyl. The term “haloalkyl” embraces radicals wherein any one or more of the alkyl carbon atoms is substituted with one or more halo groups, preferably selected from bormo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. A dihaloalkyl group, for example, may have two bromo atoms, such as a dibromomethyl group, or two chloro atoms, such as a dichloromethyl group, or one bromo atom and one chloro atom, such as a bromochloromethyl group. Examples of a polyhaloalkyl are trifluoromethyl, 2,2,2-trifluoroethyl, perfluoroethyl and 2,2,3,3-tetrafluoropropyl groups. The terms “alkoxy” and “alkoxyalkyl” embrace linear or branched oxy-containing radicals having alkyl portions of one to about ten carbon atoms, such as methoxy group. The “alkoxy” or “alkoxyalkyl” radicals may be further substituted with one or more halo atoms, such as fluoro, chloro or bromo, to provide haloalkoxy or haloalkoxyalkyl groups. The term “aralkyl” is exemplified by “phenalkyl” of which benzyl is a specific example.

Specific examples of alkyl groups are methyl, ethyl, n-propyl, isopropyl, n-butyl, sec-butyl, iso-butyl, tert-butyl, n-pentyl, iso-pentyl, methylbutyl, dimethylbutyl and neopentyl.

Included within the family of compounds of Formulas I and II are the isomeric forms of the described compounds including diastereoisomers, and the pharmaceutically-acceptable salts thereof. The term “pharmaceutically-acceptable salts” embraces salts commonly used to form alkali metal salts and to form addition salts of free acids or free bases. Since the compounds of Formulas I and II contain basic nitrogen atoms, such salts are typically acid addition salts or quaternary salts. The nature of the salt is not critical, provided that it is pharmaceutically acceptable, and acids which may be employed to form such salts are, of course, well known to those skilled in this art. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. All of these salts may be prepared by conventional means by reacting, for example, the appropriate acid or base with the corresponding compound of Formulas I and II.

The term “prodrug”, as used herein, embraces compounds which are precursors of glycine B partial agonists. Such precursor compounds can release the glycine B partial agonist by some chemical or enzymatic reaction taking place in the body or, optimally, in the brain.

Compounds of Formula I and Formula II can be synthesized by methods described in the literature. For example, syntheses of N-acyl derivatives and Schiff-base derivatives of D-cycloserine are described by N. P. Jensen et al, J. Med. Chem., 23 6-8 (1980). Syntheses of N,N′-diacyl derivatives of cycloserine are described by J. C. Howard, J. Org. Chem., 46, 1720-1723 (1981). Syntheses of alkyl derivatives of cycloserine are described by C. H. Stammer, J. Med. Chem., 13(6), 1013 (1970). Synthesis L- and D-isomers of cycloserine, as well as analogues thereof, are described by Pl. A. Plattner et al, Helv. Chim. Acta., 40, 1531 (1957).

### BIOLOGICAL EVALUATION

### Assay A: Glycine Binding Assay Procedure

Synaptic plasma membranes (SPM) were prepared from rat forebrain and stored as previously described [J. B. Monahan and J. Michel, J. Neurochem., 48, 1699-1708 (1987)]. Frozen membranes were thawed and diluted 1:20 with 0.04% triton X-100 in 50 mM tris/acetate (pH 7.4). Following incubation at 37° C. for 30 min., the SPM were collected by centrifugation at 95,000×g for 15 min. The pellet was resuspended in 50 mM tris/acetate (pH 7.4, triton-free) and handhomogenized five times. The membranes were again centrifuged as above. The pellet was washed two additional times with 50 mM tris/acetate (without homogenization) and centrifuged. The final pellet was resuspended with homogenization in 50 mM tris/acetate.

In the general receptor binding assay procedure, 10 nM [³H]glycine was added to the appropriate concentration of the test compounds and the assay initiated by the addition of 0.2-0.4 mg of ice cold SPM. The assay, which was done in 1.5 ml centrifuge tubes, was adjusted to a total volume of 1.0 ml with all additions being made in 50 mM tris/acetate, pH 7.4 at 4° C. After a 10 minute incubation at 2° C., the samples were centrifuged for 15 min. at 12,000 g (4° C.) in a Beckman Microfuge 12. The supernatant was aspirated and the tube tip containing the pelleted membranes cut off and agitated in 0.5 ml of Beckman BTS-450 tissue solubilizer for a minimum of 6 hours at room temperature. Beckman MP scintillation cocktail (5 ml) containing 7 ml/liter acetic acid was then added and the samples counted on a Beckman LS 5800 liquid scintillation counter with automatic corrections for quenching and counting efficiency. Nonspecific binding was defined as the residual binding in the presence of 0.1 mM glycine and usually amounted to 25-35% of the total binding. The binding of [³H]glycine to the SPM was analyzed using Scatchard and Hill transformations and the Kᵢ for other compounds was determined using logit-log analysis. Calculations and regression analysis were performed using templates developed for Lotus 123 as previously described.

| Result | Kᵢ (μM) |
|---|---|
| Glycine | 0.18 |
| D-cycloserine | 1.92 |
| L-cycloserine | >100 |

### Assay B: TCP Modulation Assay

[³H]TCP binding was performed using Triton X-100 washed synaptic plasma membranes (SPM) prepared from rat forebrain (30-45 day old, male SpragueDawley; Sasco, St. Charles, Mo.) as described previously [J. W. Thomas, W. F. Hood, J. B. Monahan, P. C. Contreras and T. L. O'Donohue, Brain Res., 442, 396-398 (1988)]. The assay was initiated by the addition of SPM (0.15-0.25 mg) to an incubation containing 2.0 nM [3H]TCP (47.1 Ci/mmole; New England Nuclear, Boston, Mass.) and varous concentrations of the appropriate test compound in a total volume of 0.5 ml (all additions were made in 5 mM Tris/HCl buffer, pH 7.4) and continued for 60 min at 25° C. The samples were then filtered through glass fiber filters (Schleicher and Schuell #32) which were pretreated with 0.05% (v/v) polyethylenimine. The filters were washed and the radioactivity quantitated by liquid scintillation spectrometry. Stimulation of [3H]TCP binding was measured as an increase in basal specific binding (basal binding=2583±381 DPM and this value increased to a maximum of 4712±779 DPM in the presence of 0.6 μM glycine) with nonspecific binding as the residual binding in the presence of 60 μM PCP (562±30 DPM). The K_{d} for [³H]TCP under basal conditions was 44 nM. The EC₅₀ values for the stimulation of [³H]TCP binding were determined using a four parameter logistic regression analysis.

D-cycloserine stimulates basal [³H]TCP binding in a dose dependent manner with an EC₅₀=19.7 μM. Previous data show that D-cycloserine interacts with the NMDA-associated [³H]glycine recognition site (Kᵢ=2.33±0.29 μM). No affinity for the NMDA recognition site, however, was detected as evidenced by the lack of displacement of NMDA-specific L-[3H]glutamate binding (Kᵢ>100 μM). This finding indicates that D-cycloserine enhances [³H]TCP binding through its interaction with the NMDA receptor-associated glycine recognition site (herein defined as the “Glycine B receptor”). The maximal stimulation produced by D-cycloserine, however, was significantly less than that produced by both glycine and D-serine.

This apparent lower efficacy indicates the potential partial agonist character of D-cycloserine which was confirmed by the following experiment. In the absence of exogenously added glycine, D-cycloserine has agonist properties and stimulates [³H]TCP binding to a maximum of 40-50% of the stimulation induced by glycine alone. However, in the presence of various concentrations of glycine (0.1-0.6 μM), D-cycloserine has an apparent antagonist character and reduces the maximal level of glycine stimulation. From data developed to provide a family of D-cycloserine dose-response curves (generated in the presence of several fixed concentrations of glycine), it has been observed that such dose-response curves asymptotically approach 40-50% of the maximal stimulation induced by glycine alone, a pattern characteristic of compounds with partial agonist properties as is known with different compounds acting on other receptors.

Further confirmation of the partial agonist character of D-cycloserine was demonstrated in experiments wherein a glycine dose-response analysis was performed in the presence of several fixed concentrations of D-cycloserine (0-100 μM). D-cycloserine potentiated the glycine stimulation of [³H]TCP binding at glycine concentrations below 0.1 μM, while at higher glycine concentrations (0.1-15 μM) D-cycloserine produced a rightward shift in the dose-response curve. These results are again consistent with partial agonist characteristics.

### Assay C: Post-Traumatic Memory Impairment Study

Objectives

The primary objective of this pilot study was to assess in patients with post-traumatic memory impairment: 1) The effect of a single 15 mg dose of cycloserine using the Wechsler Memory Scale (WMS) [D. Wechsler, Wechsler Memory Scale, Manual., Psychological Corporation, New York (1945)]; and 2) The effect of 5 multiple doses of 15 mg of cycloserine over 72 hours using the Memory Assessment Scales (MAS) [J. M Williams, The Memory Assessment Scales., Pyschological Assessment Resources, Odessa, Fla. (1991)]. The secondary objective of this study was to evaluate the safety of cycloserine in patients with traumatic brain injury.

Materials and Methods

The study was conducted by six investigators at six investigational sites in the United States. This was a multicenter, randomized, doubleblind, parallel group, placebo-controlled study in headtrauma patients who were experiencing significant memory disorder. Patients were randomized to receive placebo or 15 mg doses of cycloserine for a total of five equally spaced doses (morning and evening) over a 72-hour period. Safety and efficacy measurements were obtained at the screening and on Days 1 through 4 of the study. A sufficient number of patients were enrolled in the study to assure that 60 evaluable patients (30 in the cycloserine treatment group and 30 in the placebo treatment group) would complete the study.

Patients considered for enrollment were to have sustained closed head injury (ICD-9-CM#800) and post-traumatic memory impairment established by clinical examination supported by the Hahnemann Orientation and Memory Examination (HOME) [J. M. Williams, In: C. J. Long, L. Ross and M. Mutchnick, eds. Traumatic Brain Injury, Plenum, N.Y. (1990)]. The Anterograde Amnesia score was to be between 0 and 21 inclusive and the patient was to have sufficient cognitive recovery to complete the Hahnemann Memory Screening Test (MST) with a score of at least 9 [J. M. Williams, Ibid.]. Patients were to be at least 18 years of age, weighing between 40 and 100 kg, and female patients could not be at risk of pregnancy. Patients were to have a stable medical condition following the traumatic injury and be judged by the investigator to have adequate nutritional status. [Patients could have other traumatic injuries provided they were under treatment and there was no clinically significant infection or general sepsis.]

Criteria for exclusion from the study included current or history of significant systemic disorder, brain disorder, or any neurological disorder with cognitive implications other than traumatic brain injury (e.g., brain tumor). Also excluded were patients with current or history of diagnosed epilepsy or documented convulsions, or clinically significant abnormalities of thyroid function, folic acid or B₁₂. Clinically significant cardiovascular, renal, pulmonary, hepatic, gastrointestinal, infectious or hematological illness could preclude patient participation in the study (judgment of the Investigator). Concomitant medication, or medication given within seven days prior to the start of the study could preclude patient participation in the study (judgment of the Investigator and/or Clinical Monitor). Inability to follow study procedures, poor comprehension of study language, uncorrectable loss of hearing or eyesight which would interfere with study participation excluded patients from the study. Patients who had received an acridine derivative within 90 days prior to enrollment or another investigational drug within 30 days enrollment or who were known to have a hypersensitivity to cycloserine or related compounds were also excluded. This study also excluded patients taking or expected to take Hydergine, cerebral vasodilators, nootropics, choline, lecithin or amino acids. Clinically significant laboratory values outside the normal range or previous admission to the study excluded patients from participation in the study.

All study medication was provided by G. D. Searle & Co., Skokie, Ill. The unit dose of medication consisted of one white, round, biconvex 6 mm tablet containing 15 mg of cycloserine, Lot No. RCT 9384, or matching placebo, placed in a sealed sachet. Each sachet also contained a small bag of silica gel desiccant. Patients and study staff were to be warned that the desiccant was not to be ingested.

The effectiveness of a single 15 mg dose of cycloserine was assessed using the Wechsler Memory Scale (WMS) which was administered prior to the first dose of study medication and again 90 minutes after the first dose of study medication. Primary variables for the WMS are Logical Memory and Visual Reproduction. The effectiveness of a total of 5 multiple doses of 15 mg of cycloserine was assessed using the Memory Assessment Scale (MAS) which was administered prior to the first dose of study medication and again on the last day of dosing 60 minutes after the last dose of study medication. Primary variables for the MAS are Short Term Memory, Verbal Memory and Visual Memory.

Wechsler Memory Scale (WMS) and Memory Assessment Scale (MAS)

Analyses of the WMS and MAS scores utilized the primary variables listed above. In all cases the analyses used standardized scores for age or age by education rather than raw scores. Baseline characteristics for the WMS and MAS variates are described by means, standard deviations and ranges. Baseline comparability for these measures are assessed using analysis of variance, stratifying by patient type. For each primary variable treatments are compared using analysis of Invariance with each patient's baseline score taken as a covariant. Preliminary analysis also included patient type (in-patient or out-patient), baseline-by-treatment interaction and treatment-by-patient-type interaction. Non-significant terms were dropped from the model. The effects of several baseline variables on endpoint values, and change from baseline of primary variables were explored using stepwise regression. In addition, change from baseline of subscale scores of the WMS and MAS were evaluated in separate multivariate analysis of variance models. The memory quotient score of the WMS and the global memory score of the MAS were analyzed by the same met?hods as the primary WMS and MAS variables.

Seventeen in-patients (nine in the cycloserine treatment group and eight in the placebo treatment group) and 50 out-patients were enrolled in the study. At two study sites only in-patients were recruited, while primarily out-patients were recruited at the other four study sites. Time since injury ranged from three days to three years (mean=82 days) for in-patients and from 33 days to 27 years (mean=4.2 years) for out-patients. In general, there were no indications of any baseline differences in patient characteristics between treatment groups (p>0.2). However, when analyses were performed stratifying by study site, statistically significant differences were found for pre-morbid IQ and height. Patients randomized to the cycloserine treatment group had a higher pre-morbid IQ (p=0.033) and were shorter (p=0.026) than patients randomized to the placebo treatment group.

Wechsler Memory Scales

The mean baseline scores for Logical Memory and Visual Reproduction were 8.48 (s.d.=3.71) and 7.89 (s.d.=3.58), respectively. The placebo and cycloserine treatment group baseline scores were similar for Logical Memory (p=0.42). However, for Visual Reproduction, the mean value for baseline scores for cycloserine patients was somewhat higher at baseline than the mean value for baseline scores for placebo patients, although the difference was not statistically significant (p=0.069). When the Wechsler Memory Scales were repeated after patients had received one dose of study medication (Day 2), the Logical Memory and Visual Reproduction scores, corrected for baseline, did not differ significantly between the two treatment groups (p=0.72 and p=0.59, respectively). Table I presents the means and standard deviations for Logical Memory and Visual Reproduction scores for the placebo and cycloserine treatment groups at baseline and after one dose of study medication (Day 2).

TABLE I
Primary Wechsler Memory Scale Variables by Treatment

| | | Placebo | | D-Cycloserine | |
|---|---|---|---|---|---|
| | | Mean | S.D. | Mean | S.D. |
| Logical Memory | Baseline | 8.09 | (4.00) | 8.88 | (3.41) |
| | Day 2 | 8.06 | (3.69) | 8.24 | (2.81) |
| Visual Reproduction | Baseline | 7.09 | (3.85) | 8.70 | (3.14) |
| | Day 2 | 7.97 | (4.61) | 8.42 | (3.27) |

Memory Assessment Scales

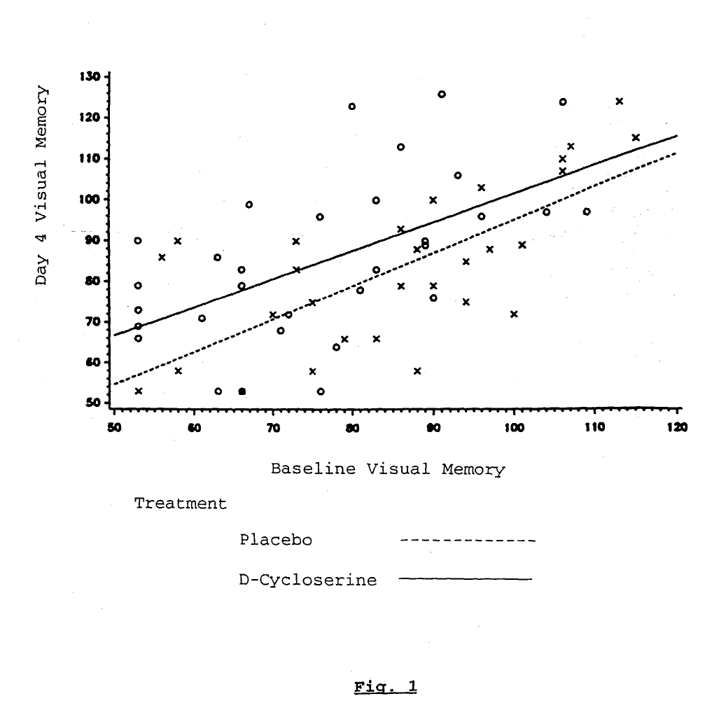

The mean baseline scores for Short Term Memory and Verbal Memory and Visual Memory were 83.5 (s.d.=15.2), 76.6 (s.d.=15.0) and 79.3 (s.d.=17.8), respectively. There were no (p>0.6) treatment differences for the primary MAS variables at baseline. In-patient and out-patient baseline differences were statistically significant for Short Term Memory (p=0.013) and Verbal Memory (p=0.003). When the Memory Assessment Scales were repeated after patients had received all five doses of study medication (Day 4), the Visual Memory scores were significantly higher for cycloserine patients than for placebo patients (p=0.023). The actual mean Visual Memory score for cycloserine patients was 87.5 compared to 78.7 for placebo patients. [These are least-square means from analysis of Invariance, corrected for baseline differences, and therefore are not exactly equal to the values given in Table II.) Furthermore, the cycloserine treatment group showed a mean change from baseline of 8.54 compared to a mean change from baseline of −1.50 for placebo treatment group. The cycloserine and placebo treatment groups were not significantly different for Verbal Memory (p=0.31) or for Short Term Memory (p=0.72). Post-drug values for Visual Memory are plotted against the corresponding baseline values in FIG. 1. Table II presents the means and standard deviations for Verbal Memory, Visual Memory and Short Term Memory scores for the placebo and cycloserine treatment groups at baseline and after five doses of study medication (Day 4).

TABLE II
Primary Memory Assessment Scale Variables by Treatment

| | | Placebo | | D-Cycloserine | |
|---|---|---|---|---|---|
| | | Mean | S.D. | Mean | S.D. |
| Short Term Memory | Baseline | 85.3 | (15.4) | 81.7 | (15.1) |
| | Day 4 | 89.4 | (15.2) | 85.9 | (18.3) |
| Verbal Memory | Baseline | 75.0 | (12.9) | 78.2 | (16.9) |
| | Day 4 | 77.0 | (16.9) | 75.8 | (14.1) |
| Visual Memory | Baseline | 81.5 | (19.5) | 77.2 | (16.1) |
| | Day 4 | 80.8 | (20.6) | 85.5 | (19.6) |

Results

Traumatic brain injury is associated with severe neuronal cell death and injury. The pattern of neuronal loss is unique for each case; some patients have numerous and widespread areas of lesion and others have relatively small lesions that involve minimal cell death. Since neurons that utilize amino acid neurotransmitters are distributed throughout cortical and subcortical areas, a partial agonist of the NMDA receptor, such as D-cycloserine, should modulate the intercommunication of these neurons with others and have a generally palliative effect on neurons that were injured by the brain trauma. As a consequence, patients with cognitive impairment resulting from lesions in virtually any part of the brain should experience amelioration of their symptoms after taking D-cycloserine. This study examined memory function in particular because NMDA receptor mechanisms have been associated with memory function, patients with traumatic injury typically have memory disorder, and previous studies found D-cycloserine to be effective with scopolamine-induced memory disorder.

The instruments used in this study included measures of immediate recall and consolidation within the verbal and visual-spatial domains. Memory recovery following a single 15 mg cycloserine dose and a five-dose regimen of cycloserine were examined. One key study measure, Visual Memory, demonstrated statistically significant, positive change following the five-dose regimen of cycloserine. The expected results included positive changes in all areas of memory, including Short-term Memory, Verbal Memory and Visual Memory. Since visual memory was the only parameter with subsequent positive findings, it is possible that this has occurred because visual memory involves a different brain pathway, which may have been affected by cycloserine.

Administration of compounds within Formulas I and II to humans can be by any technique capable of introducing the compounds into the bloodstream of a human patient, including oral administration, and by intravenous, intramuscular and subcutaneous injections.

Compounds indicated for human therapy will preferably be administered in a daily dose generally in a range, depending upon patient condition and symptomology, which is an amount therapeutically effective at the lowest possible dose, e.g., about 0.07 mg to about 0.7 mg per kilogram of body weight per day. A more preferred dosage will be a range from about 0.07 mg to about 0.4 mg per kilogram of body weight. Most preferred is a dosage in a range from about 0.1 to about 0.3 mg per kilogram of body weight per day with a dosage of about 0.2 mg per kilogram of body weight being most highly preferred. A suitable dose can be administered in multiple sub-doses per day. These sub-doses may be administered in unit dosage forms. Typically, a dose or sub-dose may contain from about 5 mg to about 50 mg of active compound per unit dosage form per day. A further preferred dosage will contain from about 5 mg to about 25 mg of active compound per unit dosage form per day. A more preferred dosage will contain from about 10 mg to about 20 mg of active compound per unit dosage per day. Most preferred is a dosage form containing about 15 mg of active compound per unit dose per day.

The active compound is usually administered in a pharmaceutically-acceptable formulation. Such formulations may comprise the active compound together with one or more pharmaceutically-acceptable carriers or diluents. Other therapeutic agents may also be present in the formulation. A pharmaceutically-acceptable carrier or diluent provides an appropriate vehicle for delivery of the active compound without introducing undesirable side effects. Delivery of the active compound in such formulations may be by various routes including oral, nasal, topical, buccal and sublingual, or by parenteral administration such as subcutaneous, intramuscular, intravenous and intradermal routes.

Formulations for oral administration may be in the form of capsules containing the active compound dispersed in a binder such as gelatin or hydroxypropylmethyl cellulose, together with one or more of a lubricant, preservative, surface-active agent or dispersing agent. Such capsules or tablets may contain a controlled-release formulation as may be provided by a dispersion of active compound in hydroxypropylmethyl cellulose.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration.

Although this invention has been described with respect to specific embodiments, the details of these embodiments are not to be construed as limitations. Various equivalents, changes and modifications may be made without departing from the spirit and scope of this invention, and it is understood that such equivalent embodiments are part of this invention.

What I claimed is:

1. A method to treat memory impairment or dificit, following or associated with Traumatic Brain Injury, by administering to a subject susceptible to or suffering from said TBI-associated memory impairment or deficit, a therapeutically-effective amount of a glycine B partial agonist.

2. The method of claim 1 wherein said glycine B partial agonist is provided by a compound, or a prodrug thereof, selected from the family of compounds of Formula I: 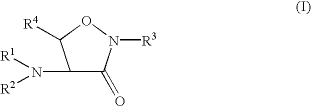
wherein R¹ is selected from hydrido, alkyl, haloalkyl, alkoxyalkyl, cycloalkyl, aralkyl and aryl; wherein each of R² and R³ is independently selected from hydrido, alkyl, aralkyl, aryl, 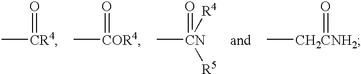
wherein R¹ and R² may be taken together to form a Schiff-base derived group selected from derivatives of aldehydes and ketones; wherein each of R⁴ and R⁵ is independently selected from hydrido, alkyl, haloalkyl, alkoxyalkyl, cycloalkyl, aralkyl and aryl; or a pharmaceutically-acceptable salt thereof.

3. The method of claim 2 wherein R¹ is selected from hydrido, lower alkyl, haloalkyl, cycloalkyl, alkoxyalkyl, phenalkyl and phenyl; wherein each of R² and R³ is independently selected from hydrido, lower alkyl, phenalkyl, phenyl, 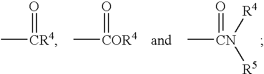
wherein said Schiff-base derived group, formed from R² and R³ taken together, is derived from acetylacetone, salicylaldehyde, benzophenbne derivatives and acetylacetic acid esters; and wherein each of R⁴ and R⁵ is independently selected from hydrido, lower alkyl, phenyl and benzyl; or a pharmaceutically-acceptable salt thereof.

4. The method of claim 3 wherein R¹ is hydrido; wherein each of R² and R³ is independently selected from 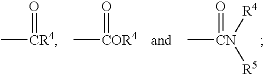
wherein said Schiff-base derived group, formed from R² and R³ taken together, is selected from 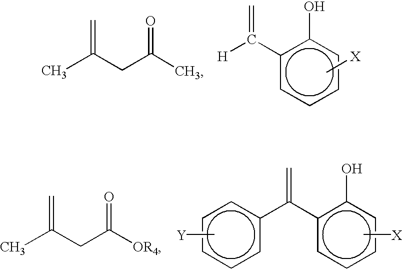
wherein each of X and Y is independently one or more groups selected from hydrido, lower alkyl and halo; and wherein each of R⁴ and R⁵ is independently selected from hydrido, lower alkyl and phenyl or a pharmaceutically-acceptable salt thereof.

5. The method of claim 4 wherein each of R¹, R², R³ and R⁴ is hydrido; and wherein said Schiff-base derived groups, formed from R² and R³ taken together, is selected from 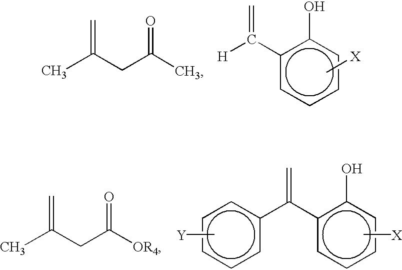
wherein each of X and Y is independently selected from fluoro, chloro and bromo; or a pharmaceutically-acceptable salt thereof.

6. The method of claim 5 wherein said compound is D-4-amino-3-isoxazolidone or a pharmaceutically-acceptable salt thereof.

7. The method of claim 6 wherein said D-4-amino-3-isoxazolidone compound is administered in a dose in a range from about 5 mg to about 50 mg of said compound per unit dosage form per day.

8. The method of claim 7 wherein said D-4-amino-3-isoxazolidone compound is administered in a dose in a range from about 5 mg to about 25 mg of said compound per unit dosage form per day.

9. The method of claim 8 wherein said D-4-amino-3-isoxazolidone compound is administered in a dose in a range from about 10 mg to about 20 mg of said compound per unit dosage form per day.

10. The method of claim 9 wherein said D-4-amino-3-isoxazolidone compound is administered in a dose of about 15 mg of said compound per unit dosage form per day.

11. A therapeutic method for treating memory impairment associated with post-traumatic brain injury in a subject when such therapy is indicated, comprising administering to said subject a therapeutically-effective amount of D-4-amino-3-isoxazolidone or a pharmaceutically-acceptable salt thereof.
